(19) United States
(12) Patent Application Publication
(10) Pub. No.: US 2022/0040491 A1
(43) Pub. Date: Feb. 10, 2022

(54) Methods and Systems for Non-Invasive Focalized Deep Brain Stimulation

(71) Applicant: Northeastern University, Boston, MA (US)

(72) Inventors: Nian-Xiang Sun, Winchester, MA (US); Mohsen Zaeimbashi, Somerville, MA (US)

(21) Appl. No.: 17/444,798

(22) Filed: Aug. 10, 2021

Related U.S. Application Data
(60) Provisional application No. 63/063,643, filed on Aug. 10, 2020.

(51) Int. Cl. A61N 2/00; A61N 2/02

(52) U.S. Cl. CPC A61N 2/006; A61N 2/02

(57) ABSTRACT

Systems and methods for providing brain stimulation (e.g., deep brain stimulation) are provided. A brain stimulation method includes applying a first magnetic field at a first location external of a brain, the first magnetic field having a waveform of a first frequency. The method further includes applying a second magnetic field at a second location external of the brain, the second magnetic field having a waveform of a second frequency. The second frequency is different from the first frequency such that temporal interference is generated at a focal point internal to the brain.

### RELATED APPLICATION(S)

This application claims the benefit of U.S. Provisional Application No. 63/063,643, filed on Aug. 10, 2020. The entire teachings of the above application are incorporated herein by reference.

### BACKGROUND

Transcranial Magnetic Stimulation (TMS) is an FDA-approved technique that has been used to intervene with malfunctioning brain circuits and has changed the way neural disorders are treated and understood. TMS is a noninvasive brain stimulation technique, does not require surgery, and does not inflict physical damage to the brain. TMS has provided relief to patients with Parkinson's disease, essential tremor, dystonia, and other debilitating disorders.

However, TMS is limited in in that it cannot provide deep brain stimulation (DBS). This is due to the rapid attenuation of a magnetic field generated by a TMS coil, leading to a maximum effective stimulation depth of around 2-3 cm beneath the scalp. The magnetic field is weak beyond this range and cannot target the central part of the brain for treatment of movement disorders such as Parkinson's disease, essential tremor, and dystonia. Furthermore, despite recent efforts to redesign TMS coils and coil geometry to improve focality and stimulation depth, TMs provides poor spatial resolution. This is due to the large size of the TMS coil, which impacts a large area of the brain, including undesirable regions, and leads to side effects, such as headache, twitching of facial muscles, or lightheadedness.

Another FDA-approved technique for brain stimulation utilizes implantable electrodes to target a specific region of the brain. This procedure, even though effective for DBS, requires surgery and hardware implantation. Site infection remains one of the most serious and worrisome problems associated with lead electrode implantation, a problem with a rate of about 15%. Furthermore, electrode implantation is an invasive procedure that requires anesthesia, opening holes in the skull, and surgery, which further complicate the use of this technique.

### SUMMARY

Systems and methods for performing noninvasive deep brain stimulation are provided. Such systems can provide for high-resolution and focalized deep brain stimulation.

A brain stimulation method includes applying a first magnetic field at a first location external of a brain, the first magnetic field having a waveform of a first frequency. The method further includes applying a second magnetic field at a second location external of the brain, the second magnetic field having a waveform of a second frequency. The second frequency is different from the first frequency such that temporal interference is generated at a focal point internal to the brain. The first location and the second location can be diametrically opposed with respect to the focal point.

A brain stimulation system includes a first magnetic coil configured to produce a first magnetic field having a waveform of a first frequency and a second magnetic coil configured to produce a second magnetic field having a waveform of a second frequency. The second frequency is different from the first frequency such that temporal interference is generated at a focal point internal to a brain disposed between the first and second magnetic coils. The system can further include a controller configured to control at least one of voltage and current to the first and second magnetic coils to produce the first and second magnetic fields. The first magnetic coil and the second magnetic coil can be configured to be worn on a head of a subject. For example, the first and second magnetic coils can be arranged in diametrically opposed positions with respect to the focal point.

The first magnetic field and the second magnetic field can be high-frequency magnetic fields to which neurons are nonresponsive. For example, the first and second frequencies can be about 1 kHz to about 1 MHz, or about 1 kHz to about 500 kHz, or about 100 kHz.

The second frequency can differ from the first frequency by a frequency that produces a beat frequency, or low-frequency envelope, to which the neurons are responsive. For example, the second frequency can differ from the first frequency by about 0.001 Hz to about 1000 Hz, or by about 1 Hz to about 500 Hz, or by about 100 Hz. The temporal interference can generate a low-frequency waveform to which neurons are responsive. The low-frequency waveform can have a frequency of about 0.001 Hz to about 1000 Hz, or about 1 Hz to about 500 Hz, or about 100 Hz. The low-frequency waveform can have an amplitude of about 0.1 mT to about 10 T, or of about 1 Oe to about 100 kOe, or up to about 100 kG.

An amplitude of the second frequency can differ from an amplitude of the first frequency to adjust a location of the focal point. For example, by adjusting the second amplitude to be lower than the first amplitude, the focal point can be adjusted to be closer to the second magnetic coil, and vice versa.

The focal point can be a deep brain region. For example, the focal point can be at or near the thalamus, subthalamic nucleus, or globus pallidus of the brain. The focal point can be adjusted to any brain structure by adjusting a location of the magnetic coils, by adjusting an amplitude of at least one of the first and second magnetic fields, or by a combination thereof.

### BRIEF DESCRIPTION OF THE DRAWINGS

The patent or application file contains at least one drawing executed in color. Copies of this patent or patent application publication with color drawings will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1A is a schematic diagram of a magnetic temporal interference (MTI) system and an applied magnetic field in a model brain.

FIG. 1B is a graph of magnetic field waveforms at a central region (1) of the model brain of FIG. 1A.

FIG. 1C is a graph of magnetic field waveforms at a peripheral region (2) of the model brain of FIG. 1A.

FIG. 2 is a schematic diagram of a 3D rat brain model and an MTI coils configuration used in simulations.

FIG. 3A illustrates a magnetic field distribution inside the cross-sectioned rat brain of FIG. 2 when coil 1 is ON and coil 2 is OFF and a corresponding graph of the magnetic field at one region of the brain in the time domain.

FIG. 3B illustrates a magnetic field distribution inside the cross-sectioned rat brain of FIG. 2 when coil 1 is OFF and coil 2 is ON and a corresponding graph of the magnetic field at one region of the brain in the time domain.

FIG. 3C illustrates a magnetic field distribution inside the cross-sectioned rat brain of FIG. 2 when both of coils 1 and 2 are ON and corresponding graphs of the magnetic fields at two regions (i, ii) of the brain in the time domain.

FIG. 4A illustrates an induced electric field inside the cross-sectioned rat brain of FIG. 2.

FIG. 4B illustrates an induced electric field gradient inside the cross-sectioned rat brain of FIG. 2.

FIG. 4C is a graph of the induced electric field along the y-axis illustrated in FIG. 4A.

FIG. 4D is a graph of the induced electric field gradient along the y-axis illustrated in FIG. 4A.

FIG. 4E is a graph of an induced electric field gradient of repetitive magnetic temporal interference (rMTI) where a gradient pulse is applied to the brain every second.

FIG. 4F is a graph of induced electric fields along the y-axis illustrated in FIG. 4A at different carrier frequencies.

FIG. 4G is a graph of induced electric field gradients along the y-axis illustrated in FIG. 4A at different carrier frequencies.

FIG. 4H is a graph of induced electric fields at the center of the brain of FIG. 4A in the time domain at different carrier frequencies.

FIG. 5A illustrates an induced electric field distribution inside the cross-sectioned rat brain of FIG. 2 when current applied to coil 1 and coil 2 are equal.

FIG. 5B illustrates an induced electric field distribution inside the cross-sectioned rat brain of FIG. 2 when current applied to coil 2 is two times that of current applied to coil 1.

FIG. 5C illustrates an induced electric field distribution inside the cross-sectioned rat brain of FIG. 2 when current applied to coil 2 is four times that of current applied to coil 1.

FIG. 5D is a graph of normalized electric fields along an axis of the brain at different ratios of current applied to coils 1 and 2.

FIG. 6A is a schematic of an experimental setup of a deep brain stimulation system.

FIG. 6B is a two-dimensional (2D) plot of measurement results of an experiment for the region shown in FIG. 6A.

FIG. 6C is a time domain graph of the magnetic field produced at the center of the region shown in FIG. 6B.

FIG. 6D is a 2D plot of simulation results of the experiment for the region shown in FIG. 6A.

FIG. 6E is a time domain graph of the simulated magnetic field produced at the center of the region shown in FIG. 6D.

### DETAILED DESCRIPTION

A description of example embodiments follows.

Systems and methods for providing non-invasive, high-resolution, and focalized deep brain stimulation are provided. The systems and methods provide for a technique referred to herein as magnetic temporal interference (MTI). MTI employs a time domain interference of two high-frequency magnetic fields, which can create a localized, low-frequency envelope capable of targeting any depth inside the brain. Neural systems are non-responsive to each of the high-frequency magnetic fields alone, but a neural system can respond to a low-frequency component resulting from the interference. The low-frequency component can non-invasively stimulate a deep brain area at a high resolution without impacting peripheral regions. The provided systems and methods can enable precise and efficient brain stimulation for various neuroscience applications as well as for treatment of various neurological and neuropsychiatric disorders and diseases.

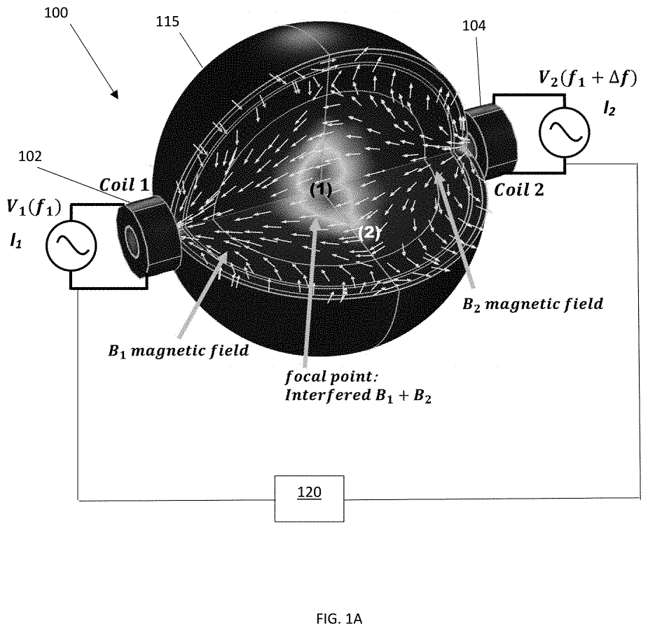

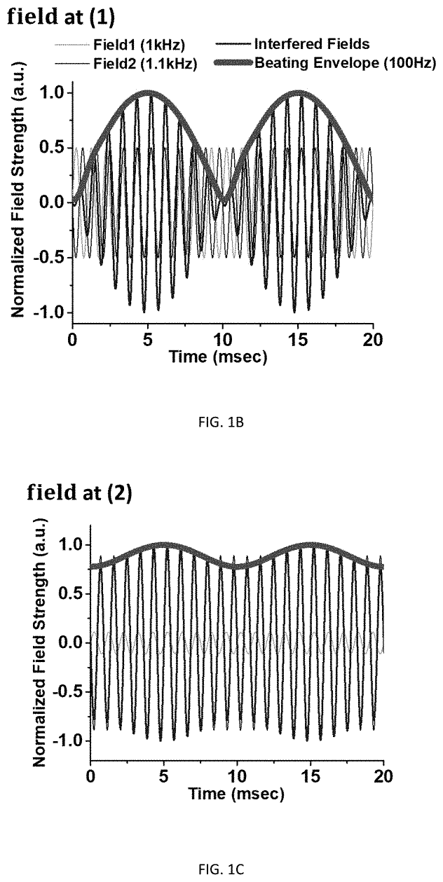

As illustrated in FIG. 1, a brain stimulation system 100 includes at least two magnetic coils 102, 104. A first magnetic coil 102 is configured to produce a first magnetic field (B₁) having a waveform of a first frequency (f₁). A second magnetic coil 104 is configured to produce a second magnetic field (B₂) having a waveform of a second frequency that differs from the first (f₁+Δf). Temporal interference is generated at a focal point (1) internal to a brain disposed between the first and second magnetic coils. The brain stimulation system 100 can further include a controller 120 configured to control at least one of voltage (V₁, V₂) and current (I₁, I₂) to the first and second magnetic coils 102, 104 to produce the first and second magnetic fields.

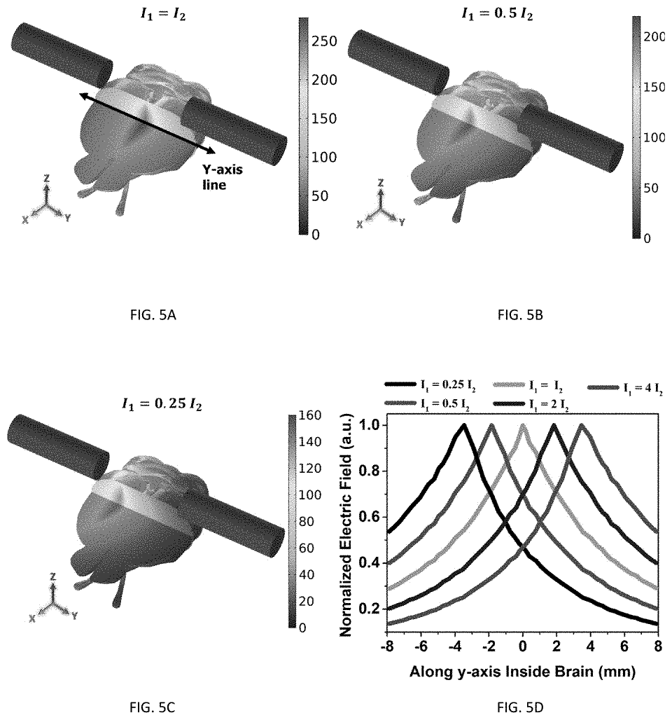

As illustrated with respect to the example system show in FIG. 1, the MTI technique relies on temporal interference of two high-frequency magnetic fields generated by two electromagnetic coils. A neural system does not respond to each of these high-frequency magnetic fields alone because of intrinsic low-pass filtering properties of neural membranes, i.e., the brain cannot follow or react to high-frequency magnetic fields (e.g., >1 kHz) created by the coils. See Hutcheon B. and Yarom Y, Resonance, oscillation and the intrinsic frequency preferences of neurons, TINS, Vol. 23, No. 5, 2000. However, if these two magnetic fields differ by a small amount in frequency (Δf), a temporally interfered signal can result that contains a low-frequency envelope (FIG. 1B), and the neural system can follow and respond to this envelope. In this technique, a peripheral area of the brain is impacted only by the high-frequency fields B₁ (ω₁) and B₂ (ω₁+Δf), which do not stimulate nerves, while the deep brain area—where the two fields interfere and create a magnetic field that contains a low-frequency envelope corresponding to Δf—can be stimulated. The focal point where the two fields interfere can be adjusted to any depth inside the brain by changing the value of the currents injected to the coils (see FIGS. 5A-5D), by changing a location of the coils, or both.

FIGS. 1A-1C further illustrate a simulation of a brain 115 having a radius of 10 cm and different layers representing skin, skull, cerebrospinal fluid (CSF), grey matter, and white matter. A focalized magnetic field beam in a deep brain area is generated without impacting the peripheral regions. In this example, Coil 1 and Coil 2 are excited with a high-frequency sine wave signal at 1 kHz and 1.1 kHz. The low-frequency envelope is the difference between the two frequencies and, in this example, is equal to 100 Hz. FIG. 1B illustrates the magnetic field waveform at region (1) in a central region of the brain 115, where the two fields interfere and create the low-frequency envelope with high amplitude (also referred to as a beat frequency). FIG. 1C illustrates the magnetic field waveform at region (2), outside the central area of the brain 115, and shows a low-frequency envelope with a small amplitude. The peripheral regions are predominantly impacted by the high-frequency fields B₁ and B₂ and, therefore, brain structures (e.g., neurons) in these regions are not stimulated. In contrast, a central region is impacted by the high amplitude, low-frequency envelope, which can result in the stimulation of brain structures in this area.

The first and second magnetic fields can be high-frequency magnetic fields to which neurons are nonresponsive. For example, the first and second frequencies can be of about 1 kHz to about 1 MHz (e.g., 0.9 kHz, 1 kHz, 500 kHz, 1.1 MHz). The second frequency can differ from the first frequency by a frequency that produces a beat frequency to which the neurons are responsive. For example, the second frequency can differ from the first frequency by about 0.001 Hz to about 1000 Hz, or by about 1 Hz to about 500 Hz, or by about 10 Hz to about 500 Hz, or by about 50 Hz to about 200 Hz, or by about 100 Hz (e.g., 90 Hz, 99 Hz, 100 Hz, 101 Hz, 110 Hz).

The temporal interference can generate a low-frequency waveform to which neurons are responsive. For example, the low-frequency waveform can have a frequency of about 0.001 Hz to about 1000 Hz, or by about 1 Hz to about 500 Hz, or by about 10 Hz to about 500 Hz, of about 50 Hz to about 200 Hz, or of about 100 Hz (e.g., 90 Hz, 99 Hz, 100 Hz, 101 Hz, 110 Hz).

The low-frequency waveform can have an amplitude of about 0.1 mT to about 10 T, or of about 1 Oe to about 100 kOe, or up to about 100 kG. An amplitude of the first or second frequency can differ from an amplitude of the other of the first and second frequencies to adjust a location of a focal point. For example, by adjusting the second amplitude to be lower than the first amplitude, the focal point can be adjusted to be closer to the second magnetic coil, and vice versa (e.g., FIGS. 5A-5D, described further below).

The magnetic coils can be configured to be worn on a head of a subject. For example, the first and second magnetic coils can be arranged in diametrically opposed positions about a subject's brain with respect to a focal point, as illustrated in FIG. 1A. The focal point can be any deep brain area, including, for example, areas at or near the thalamus, subthalamic nucleus, or globus pallidus of the brain.

A number of coils placed at the subject, a size of the coils, a number of turns of each coil, a magnetic core material of the coils, and the locations at which the coils are placed with respect to the brain can be adjusted and optimized to fulfil multiple objectives. For example, a size of the coils and the locations at which they are placed with respect to the brain can be adjusted such that the magnetic fields produced by the two coils can interfere at any given area and/or any given depth inside the brain with a high spatial resolution. Unlike TMS techniques, which can only stimulate peripheral regions of the brain and lack high spatial resolution, MTI techniques are able to focus the magnetic and electric fields at a deep brain area. In another example, the generated electric field gradient, which can be an important parameter for magnetic brain stimulation, can be adjusted. The generated electric field gradient can be higher than a threshold value required for brain stimulation, such as greater than about 11 k V/m². See Lee et al., Implantable microcoils for intracortical magnetic stimulation, Sci. Adv. 2016; and Pashut et al., Mechanisms of Magnetic Stimulation of Central Nervous System Neurons, PLoS Comp. Bio., Vol. 7:3, 2011. In another example, frequency of the applied magnetic fields can be adjusted. For example, the coils can operate efficiently at frequencies of up to about 50 kHz or up to about 100 kHz. The induced electric field of the coils increases linearly with operational frequency, providing for the generation of large electric fields. In other words, large electric fields, above required threshold values for brain stimulation, can be achieved by increasing operational frequency of the coils and without increasing current, which can significantly reduce generated heat and power consumption.

MTI techniques provided herein have multiple advantages over existing neural stimulation methods. Unlike TMS techniques, MTI can target central parts of the brain without impacting the peripheral areas as the high-frequency magnetic field generated by each MTI coil alone does not stimulate nerve tissue and as a generated low-frequency envelope can be focalized to a deep brain region.

Furthermore, a spatial resolution of the MTI technique is higher than that of TMS. TMS coils are generally large (e.g., 10-15 cm), and the field generated by the TMS coils impacts a large area of the brain, leading to undesirable side effects, such as a headache, twitching of facial muscles, or lightheadedness. With MTI, by appropriately adjusting the coils and, optionally, increasing a number of MTI coils, a size and focal point can be reduced and optimized to achieve a much higher spatial resolution.

Further still, unlike in TMS, in MTI, it is possible to apply high-frequency sine wave signals (e.g., tens of kHz) to the coils and, therefore, the induced electric field and electric field gradient generated by MTI coils can be significantly boosted. Faraday's law provides that an induced electric field and electric field gradient are linearly proportional to a rate of change of a magnetic field. This phenomenon can provide for a significant advantage of MTI over TMS techniques because threshold values of electric field and electric field gradients for brain stimulation can be achieved by increasing an operational frequency of the coils and without significantly increasing current applied to the coils. A smaller current applied to the coils can result in less heating, which is currently one of the challenges of TMS techniques. With TMS techniques, thousands of Amps of current applied to the TMS coil leads to excessive heating in the coil.

MTI also offers several advantages over implantable electrodes, primarily relating to MTI being noninvasive. Electrode implants require brain surgery and anesthesia and can inflict physical damage to the brain. In contrast, MTI coils can be placed outside the head, and the magnetic field can penetrate through the skill and brain and stimulate the neural system using the low-frequency envelope that is generated by the temporally interfered fields.

Electrical temporal interference techniques have been investigated in Grossman et al., Noninvasive Deep Brain Stimulation via Temporally Interfering Electric Fields, Cell, Vol. 169, 2017, where the authors used two pairs of electrodes to directly inject high-frequency currents to the brain. However, this technique suffers from several limitations due to the need to inject direct current flow through the skin and/or brain. The authors in Grossman et al. proposed two mechanisms to deploy this technique on human brain: 1) placing the electrodes on the skin outside the brain, in which case, because of the low electrical conductivity of the skull, most of the current applied to the electrodes will flow through the skin without actually penetrating through the brain and reaching a deep brain area; and 2) placing the electrodes under the skull and on the surface of the brain, which is requires an invasive surgery for electrode implantation. In an event that one needs to move the focal point inside the brain and stimulate a different area, relocating the electrodes and performing further surgery would be required as well using electrical temporal interference methods.

Unlike electrical temporal interference techniques, where multiple pairs of electrodes are placed under the skull and in direct contact with the brain surface, MTI does not require any electrode implantation or surgery and does not inflict any physical damage to the skull or brain.

Additional examples are provided in the following Examples.

### EXEMPLIFICATION

### Example 1. Simulation Data Validation of MTI Technique

Magnetic temporal interference techniques were simulated in COMSOL Multiphysics® software (COMSOL, Burlington Mass.). The simulation data shows that MTI can be effective for noninvasive, high-resolution, and localized deep brain stimulation.

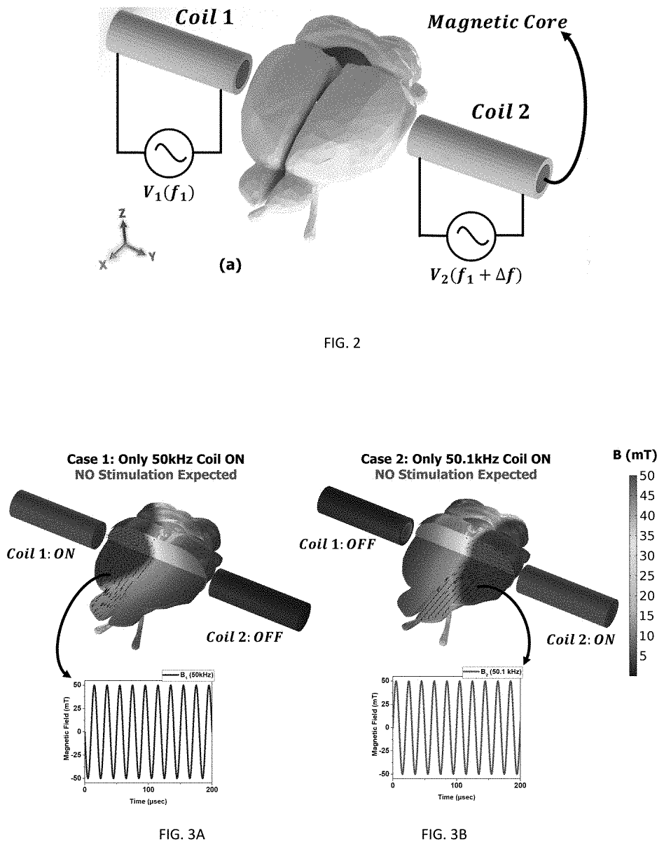

A 3D rat brain model with a size of 10×16×21 mm³ was used in the simulations. In the simulations, the two designed MTI coils produce a magnetic field, and the magnetic field penetrates through the brain and induces an electric field and electric field gradient, which can stimulate the brain neural system. The rat model and coil configuration are shown in FIG. 2. Coils 1 and 2 are excited using two different sources with a same amplitude but slightly different frequencies (Δf). The coils have Manganese-zinc ferrite (MnZn) magnetic cores with relative permeability of 17 that boosts the generated magnetic field.

An MnZn core can operate at frequencies up to 100 kHz. The pulsed sine wave current applied to the coils was 40 Amp and was simulated to run for 10 msec instead of continuous powering, thereby significantly reducing heating effects. The 10 msec timing is equivalent to one full cycle of a low-frequency envelope of 100 Hz. The currents applied to the coils can be at a 180° phase difference so as to have full destructive interference at t=0 and full constructive inference at t=5 msec.

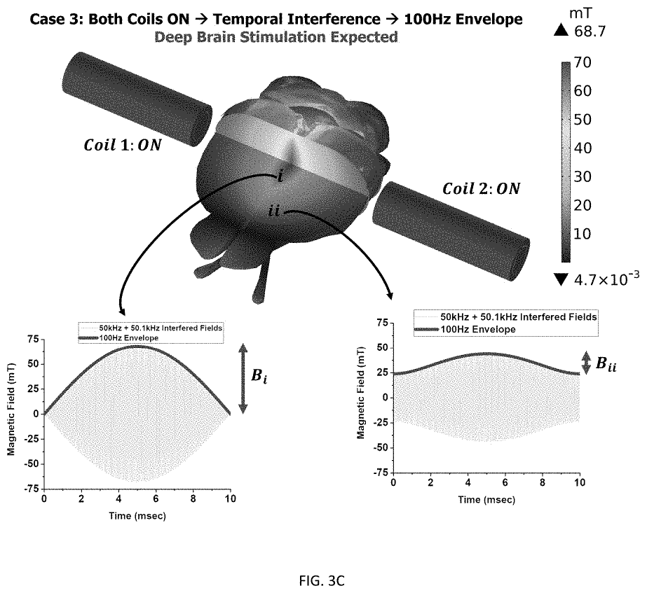

Results of the simulations are shown in FIGS. 3A-3C. FIG. 3A shows a magnetic field distribution inside a cross-section of the rat brain model when only Coil 1 (with 50 kHz frequency) is ON. As shown, the left part of the brain is impacted by this high-frequency signal, but no stimulation is expected since the frequency is too high and neurons cannot respond or follow the generated field. FIG. 3B shows a scenario where only Coil 2 (with 50.1 kHz frequency) is ON. A same situation exists here, where neurons cannot respond to this high-frequency field and, therefore, no stimulation happens. Time domain graphs of the 50 kHz and 50.1 kHz sine wave magnetic field produced by Coil 1 and Coil 2, respectively, are also shown in FIGS. 3A and 3B.

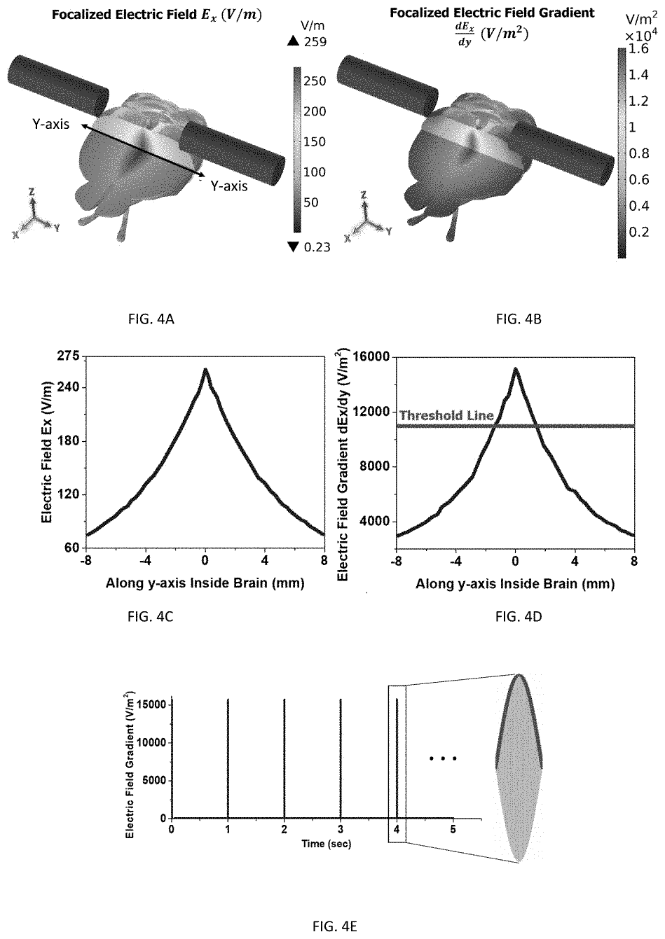

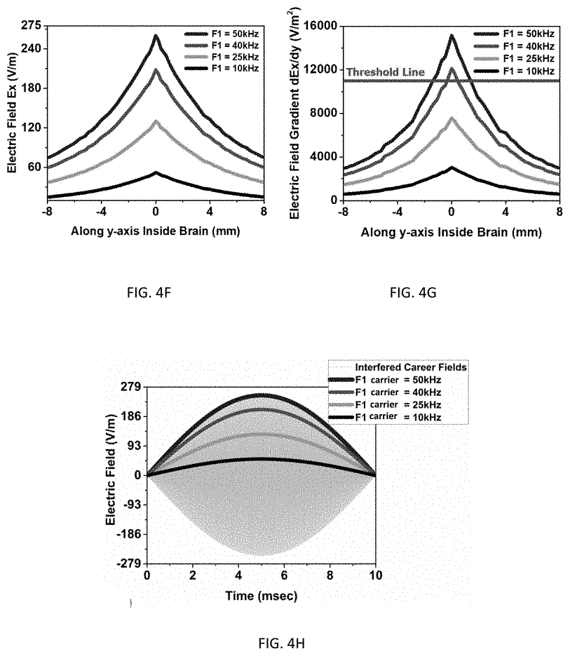

FIG. 3C shows the temporally interfered magnetic field distribution inside the cross-sectioned rat brain when both Coil 1 and Coil 2 are ON. This field distribution shows the amplitude of the low-frequency envelope (100 Hz in this case), which is at a maximum in the central part of the brain and at a minimum in a peripheral region. The neural system in the deep brain focal region can demodulate and respond to this low-frequency envelope, which is formed as a result of the temporal interference of the two fields from Coils 1 and 2. Time domain graphs of the magnetic field signal in the central and peripheral regions when both coils are ON are also shown in FIG. 4C. The amplitude of the low-frequency envelope is large at the center and small in peripheral regions.

### Example 2. Field Strength Validation of MTI for Neural Stimulation

An electric field gradient of about 11 kV/m² is known to activate neurons.

FIGS. 4A-4D show the simulation results for the generated electric field and electric field gradient by MTI coils. FIGS. 4A and 4B show the induced electric field and electric field gradient, respectively, both of which are focused in a deep brain area with a high resolution. FIGS. 4C and 4D show the electric field and electric field gradient along the y-axis line shown in FIG. 4A.

As shown, the induced electric field (Eₓ) and electric field gradient (dEₓ/dy) are focused in a central part of the brain with a high spatial resolution. The electric field gradient is at a maximum in the center and is as high as 16 kV/m², which is higher than the threshold value of 11 kV/m². The stimulated region in the deep brain area, where the electric field gradient is above the threshold value, is around 2-3 mm, which indicates that MTI has a very high spatial resolution.

FIG. 4E shows the induced electric field gradient of repetitive MTI (rMTI) where a gradient pulse is applied every second to the deep brain region. Repetitive transcranial magnetic stimulation has been extensively researched in the past two decades, and it has been widely shown that this technique can be very effective for the treatment of major depression disorder, Parkinson's disease, and stroke. See Baeken et al., Repetitive transcranial magnetic stimulation treatment for depressive disorders: current knowledge and future directions, Curr. Opin. Psychiatry, Vol. 32, 2019; Liao et al., Repetitive transcranial magnetic stimulation as an alternative therapy for dysphagia after stroke: a systematic review and meta-analysis, Clinical Rehabilitation Vol. 31(3), 2017; Magavi et al., A review of repetitive transcranial magnetic stimulation for adolescents with treatment-resistant depression, Int'l Rev. Psychiatry, Vol. 29, No. 2, 2017; and Yang et al., Repetitive transcranial magnetic stimulation therapy for motor recovery in Parkinson's disease: A Meta-analysis, Brain and Behavior, 2018.

FIGS. 4F and 4G show the induced electric field and electric field gradient along the y-axis line (shown in FIG. 4A) at different carrier frequencies f₁. As shown, the induced fields linearly increase with increasing frequency, a phenomenon that is due to Faraday's law of electromagnetics, where an induced electric field is linearly proportional to a rate of change of a magnetic field over time. This phenomenon is advantageous for MTI, where a larger induced electric field and field gradient can be achieved directly by increasing the carrier frequencies of the coils, without a need to increase current, which can significantly increase heating in the system. The spatial resolution of 2-3 mm described above can be further improved by reducing the carrier frequencies or the current such that only the desired area at the center is impacted by field gradients larger than a threshold value. In FIG. 4G, for instance, at a carrier frequency of 40 kHz, a much smaller area—around 1 mm—is impacted by field gradients greater than the threshold value. In other words, by selecting a particular carrier frequency, the spatial resolution can be improved and provided such that a defined area deep inside the brain is stimulated.

FIG. 4H shows the time domain electric field at the center of the brain at different carrier frequencies f₁. The low-frequency envelope shaped by the temporal interference of two carrier frequencies are shown. The induced electric field linearly increases by increasing carrier frequency, while a low-frequency envelope of 100 Hz is still preserved at all cases.

### Example 3. Moving the MTI Focal Point by Changing the Applied Currents to the Coils

In previous parts, the MTI technique was simulated and investigated for focusing a magnetic or electric beam deep at the center of the brain. It was shown that, using two MTI coils, the low-frequency field component can be successfully focalized precisely at the center. In this section, focalizing the field at different depths inside the brain, and not necessarily at the center, was simulated. MTI can focalize the beam at any depth inside the brain by adjusting a ratio of the currents applied to Coils 1 and 2. The equation (Eq. 1) for calculating the amplitude of low-frequency component is shown in the next experimental section. According to this formula, maximum low-frequency amplitude occurs where the amplitude of high-frequency electric (or magnetic) fields E₁ (f₁) and E₂ (f₁+Δf) are equal. When applied current to both coils are equal, shown in FIG. 5A, the field distribution E₁ (f₁) and E₂ (f₁+Δf) generated by the coils on two sides of the brain are symmetric, and, therefore, have equal value at the center of the brain. As such, the focal point is provided at a central region of the brain.

When one of the coils is excited at a larger current, however, the focalized beam can be shifted toward the coil with weaker current. In FIG. 5B, for instance, where applied current to Coil 2 is two times as large as applied current to Coil 1, the beam is shifted toward Coil 1. This occurs because Coil 1 produces a weaker electric field compared to Coil 2, and, therefore, the location where both high-frequency electric fields are equal—in other words, the location where beam focalization happens—is in the region closer to Coil 1.

FIG. 5C shows the simulation results of the applied current to Coil 2 being four times as large as the applied current to Coil 1. The focalized beam in this case is further shifted toward Coil 1 as compared to FIGS. 5A and 5B. FIG. 5D shows normalized electric fields along the y-axis inside the brain at different current ratios applied to Coil 1 and Coil 2. As described above, by changing the ratio of currents (I₁ and I₂), the MTI focal depth can be adjusted (e.g., the focal point can be shifted left or right), without changing a location of the coils or readjusting a system setup.

### Example 4. Experimental Demonstration of MTI

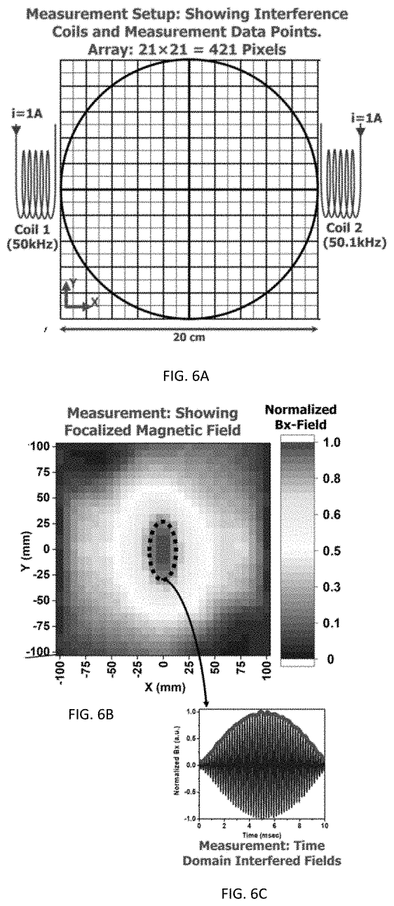

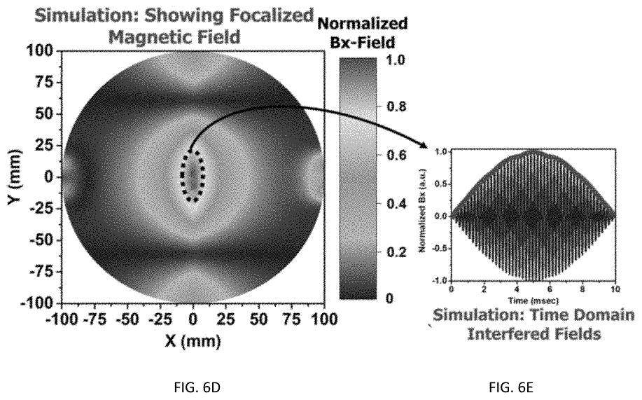

In previous sections, COMSOL simulation results are described, which were used to validate the theory, capabilities, and advantages of the MTI technique. In this section, a prototype system and experimental investigation are described. For the experiment, two homemade solenoid coils were used, each with 2 cm dimeter, 2 cm height, and 15 turns. A matching capacitor was also added in series to each coil to cancel the inductance value and to assure that a series LC circuit is in a resonance condition at 50 kHz operational frequency. Putting the circuit in resonant mode significantly reduces the reflection power and helps to achieve a maximum magnetic field from the coils. Coil 1 was excited at 50 kHz and Coil 2 at 50.1 kHz, both under a fixed current of 1 Amp. The two coils were each placed on one of two sides of a circular region with radius of 10 cm, which corresponds to the radius of the human brain model shown in FIG. 1. This circular region is shown in the diagram of FIG. 6A, where an array of 21×21 measurement data points, or 421 pixels in total, was provided. After exciting the coils under the described conditions, the amplitude of the 50 kHz and 50.1 kHz magnetic fields was measured at each pixel using a 1 mm size homemade search coil connected to a spectrum analyzer. Therefore, at each pixel, two data points were provided: Fast Fourier Transform (FFT) amplitude of magnetic field at B₁ (f₁) and at B₂ (f₁+Δf). Then, using the standard formula below, the amplitude of the low-frequency envelope was calculated:

|Bₑₙᵥₑₗₒₚₑ(\vec{r},\vec{n})|=∥FFT{\vec{B}₁(\vec{r})·\vec{n}}+FFT{\vec{B}₂(\vec{r})·\vec{n}}−|FFT{\vec{B}₁(\vec{r})·\vec{n}}−FFT{\vec{B}₂(\vec{r})·\vec{n}}∥ Eq. (1)

Based on the simulation results from previous sections, it was already known that the magnetic field low-frequency envelope is at a maximum along the x-axis, which is the axis parallel to the solenoid axis, and other field components along the y- and z-axes are significantly smaller in comparison. Thus, by carefully aligning the search coil along the x-axis, only the fields B₁ (f₁) and B₂ (f₁+Δf) were picked up along this axis, and the low-frequency envelope Bx using Eq. (1) was calculated.

It is notable that, even though this measurement was performed in air medium, it is expected that the temporal interference of the two fields will lead to the same results and same focality in a biological medium. This is because of two factors. First, the measurement region and its size are several orders of magnitude smaller than the operational wavelength (˜6000 m), and, as such, field distribution is completely inductive. If, for instance, the operational frequency were extremely high and, therefore, the wavelength was comparable to the measurement area, then the field distribution might be in a propagation mode. In such a circumstance, a temporal interference pattern and focality can be significantly impacted by adding a biological tissue or a high-conductivity material close to the coils, but this is not the case. Second, operational frequency is low and, thus, electrical conductivity of the biological tissue (e.g., brain tissue) is very close to that of air. A small electrical conductivity reduces the power dissipation in the tissue, as well as reduces the magnetic field distortion in the medium. Therefore, it can be safely concluded that the magnetic field temporal interference results at this frequency range in air are a good representation of the results achievable in a biological medium, such as human brain.

FIG. 6B shows the 2D measurement results of the normalized amplitude of the low-frequency magnetic field envelope in the region between two coils. As shown, the low-frequency magnetic beam is focused at the central region between two coils. FIG. 6C shows the time domain measurement at the central region, which includes the interfered fields and low-frequency envelope shown in red.

FIG. 6D shows the COMSOL simulation results of a same set up, coil parameters, and region size. The simulation results confirm the measurement data and show that the amplitude of low-frequency magnetic field envelope is maximum at the center of the measured area. FIG. 6E shows the time domain measurement of the simulation results at the central region, which includes the interfered fields and low-frequency envelope shown in red. Outside the central region, the interference is partial, and the low-frequency envelope was smaller than the value shown in these figures.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A brain stimulation method, comprising:
applying a first magnetic field at a first location external of a brain, the first magnetic field having a waveform of a first frequency; and
applying a second magnetic field at a second location external of the brain, the second magnetic field having a waveform of a second frequency, the second frequency being different from the first frequency such that temporal interference is generated at a focal point internal to the brain.

2. The brain stimulation method of claim 1, wherein the first magnetic field and the second magnetic field are high-frequency magnetic fields to which neurons are nonresponsive.

3. The brain stimulation method of claim 2, wherein the first and second frequencies are of about 1 kHz to about 1 MHz.

4. The brain stimulation method of claim 2, wherein the second frequency differs from the first frequency by a frequency that produces a beat frequency to which the neurons are responsive.

5. The brain stimulation method of claim 2, wherein the second frequency differs from the first frequency by about 0.001 Hz to about 1000 Hz, or by about 1 Hz to about 500 Hz.

6. The brain stimulation method of claim 1, wherein the temporal interference generates a low-frequency waveform to which neurons are responsive.

7. The brain stimulation method of claim 6, wherein the low-frequency waveform has a frequency of about 0.001 Hz to about 1000 Hz, or by about 1 Hz to about 500 Hz.

8. The brain stimulation method of claim 6, wherein the low-frequency waveform has an amplitude of about 0.1 mT to about 10 T.

9. The brain stimulation method of claim 1, wherein an amplitude of the second frequency differs from an amplitude of the first frequency.

10. The brain stimulation method of claim 1, wherein the first location and the second location are diametrically opposed with respect to the focal point.

11. The brain stimulation method of claim 1, wherein the focal point is at or near the thalamus, subthalamic nucleus, or globus pallidus of the brain.

12. A brain stimulation system, comprising:
a first magnetic coil configured to produce a first magnetic field having a waveform of a first frequency; and
a second magnetic coil configured to produce a second magnetic field having a waveform of a second frequency, the second frequency being different from the first frequency such that temporal interference is generated at a focal point internal to a brain disposed between the first and second magnetic coils.

13. The brain stimulation system of claim 12 further comprising a controller configured to control at least one of voltage and current to the first and second magnetic coils to produce the first and second magnetic fields.

14. The brain stimulation system of claim 12, wherein the first magnetic field and the second magnetic field are high-frequency magnetic fields to which neurons are nonresponsive.

15. The brain stimulation system of claim 14, wherein the first and second frequencies are of about 1 kHz to about 1 MHz.

16. The brain stimulation system of claim 14, wherein the second frequency differs from the first frequency by a frequency that produces a beat frequency to which the neurons are responsive.

17. The brain stimulation system of claim 14, wherein the second frequency differs from the first frequency by about 0.001 Hz to about 1000 Hz, or by about 1 Hz to about 500 Hz.

18. The brain stimulation system of claim 12, wherein the temporal interference generates a low-frequency waveform to which neurons are responsive.

19. The brain stimulation system of claim 18, wherein the low-frequency waveform has a frequency of about 0.001 Hz to about 1000 Hz, or by about 1 Hz to about 500 Hz.

20. The brain stimulation system of claim 18, wherein the low-frequency waveform has a maximum amplitude of about 0.1 mT to about 10 T.

21. The brain stimulation system of claim 12, wherein an amplitude of the second frequency differs from an amplitude of the first frequency.

22. The brain stimulation system of claim 12, wherein the first magnetic coil and the second magnetic coil are configured to be worn on a head of a subject, the first and second magnetic coils arranged in diametrically opposed positions with respect to the focal point.
